(19) United States
(12) Patent Application Publication
(10) Pub. No.: US 2006/0178293 A1
(43) Pub. Date: Aug. 10, 2006

(54) Cardiotonic polypeptides

(76) Inventors: Eliahu Zlotkin, Meveseret Zion (IL); Morris Mosseri, Jerusalem (IL)

Correspondence Address: NATH & ASSOCIATES, 112 South West Street, Alexandria, VA, 22314, US

(21) Appl. No.: 10/557,142

(22) PCT Filed: May 19, 2004
(86) PCT No.: PCT/IL04/00427

§ 371 Date: Mar. 13, 2006

(30) Foreign Application Priority Data
May 19, 2003 (IL) 155972

(51) Int. Cl. A61K 38/00; C07K 14/00

(52) U.S. Cl. 514/2; 536/23.5; 530/300

(57) ABSTRACT

A polypeptide which has a positive inotropic effect on cultured cardiomyocytes and is non-toxic to mammals. The polypeptide may be isolated from Buthinae scorpion venom. Also disclosed is a pharmaceutical composition comprising the polypeptide and a DNA molecule encoding the polypeptide.

### FIELD OF THE INVENTION

This invention relates to cardiotonic substances isolated from scorpion venom.

### BACKGROUND OF THE INVENTION

The following references may be helpful in understanding the background of the invention:
1. Gordon et al, (1997) Sodium channels as targets for neurotoxins: mode of action and interaction of neurotoxins with receptor sites on sodium channels, in: Toxins and Signal Transduction, Gutman and Lazarovici, eds. Harwood, Amsterdam, PP 13. 119-149.
2. Zlotkin, E. et al (1978) Chemistry and Pharmacology of Buthinae Scorpion Venom, in Arthropod Venoms, Sergio Bettini, ed. pgs. 317-369.
3. Gmpp, I. L. et al (1980) Effects of the venom of the Yellow Scorpion on the isolated work-performing guinea pig heart, Toxicon 18:261-270.
4. Lester, et al (1982) Purification, characterization and action of two insect toxins from the venom of the scorpion Buthotus Judaicus BBA 701:370-381.

The significant reduction in mortality achieved through the successful operative treatment of coronary occlusion has tremendously increased the magnitude of the Heart Failure disorder in the surviving aging population. The fundamental abnormality of the heart in the syndrome of heart failure is a diminished ability of the failing muscle to develop force and shorten at a given velocity and at specified loading conditions. The major clinical manifestations include breathlessness, fatigue and fluid retention. Coronary artery disease is the most common cause of heart failure, but systemic hypertension dilated cardiomyopathy and valvular heart disease are also common causes of chronic heart failure.

Therapeutic modalities in the treatment of heart failure should aim at improving the clinical symptoms, the patient's prognosis and preferably, both. Current treatments of heart failure include bed rest, water and salt restriction, supplemental oxygen therapy, diuretics, vasodilators (e.g. hydralazine and nitrates), ACE inhibitors (e.g. captopril and enalapril), angiotensin II-receptor blockers, beta-adrenergic receptor blockers, aldosteron and digitalis.

When dealing with chemotherapy, digitalis and inotropic agents deserve attention. Digitalis was the first and the most common drug used for congestive heart failure due to its positive inotropism and modulation of various neurohormonal factors. However, there are two important drawbacks. The first is related to its low “therapeutic index” which demands a careful drug dosing. The second corresponds to the recently completed Digoxin Investigators Group (DIG) trial which has followed 7788 patients with congestive heart failure for 5 years and concludes that digoxin did not significantly affect mortality.

Inotropic agents are substances that influence the force or energy of cardiac muscular contractions, thereby tending to affect the cardiac output. If the substance increases the force of contraction of the heart, it is considered to have a positive inotropic effect. Inotropic agents, such as Dopamine and Dobutamine were pursued during the past decades for the treatment of heart failure. These medications improve the condition of patients with severe heart failure only in the short term. Mortality end-point heart failure trials with other drugs have suggested that intervention with certain drugs (such as Milrinone and Flosequinan and Vesnarinone) actually increases mortality. It is also noteworthy that it is not clear if chronic parenteral inotropic infusion reduces end-stage heart failure mobidity and mortality, though most believe that symptoms are substantively reduced at the cost of higher sudden cardiac death rates. In general, positive inotropic drugs, that do not ameliorate adverse neurohormonal profiles seem detrimental to long-term prognosis in heart failure patients, although they may attenuate morbidity.

To summarize there is a need for a non-toxic, inotropic agent that will improve both patients' symptoms and prognosis.

Like in other venomous systems, the pharmacology of scorpion venom has been shown to be entirely based on polypeptides. The venoms of scorpions belonging to the Buthidae family possess a series of “long chain” polypeptide toxins (60-70 amino acids; 4 disulphide bridges) which affect the voltage gated sodium channels (VGSCs). These toxins are currently divided in two major categories: the alpha toxins (which affect sodium current inactivation and prolong the action potential) and beta toxins (which affect activation, shift voltage dependence towards the resting potential and induce repetitive firing of short duration action potentials). Each of the above two categories of the alpha and beta toxins can be further subdivided into those which affect mammals and those which are insect specific and are non toxic to mammals.

The beta category includes the excitatory and depressant insect selective neurotoxins [1]. The alpha scorpion toxins are currently subdivided into several groups according to their binding specificity, electrophysiology, distinction between vertebrate and insect sodium channels and vertebrate central and peripheral sodium channels. The neurotoxic and lethal effects of scorpion venom to mammals are attributed mainly to the alpha neurotoxins' affecting the voltage gated sodium channels.

When dealing with the pathophysiology of scorpion venom in general, two mechanisms of action may be discerned: (1) an indirect mechanism mediated by the nervous system, and (2) a direct mechanism independent of the nervous system, which involves the direct interaction of the venom components with muscle (skeletal, smooth, cardiac) cell membranes [2].

It may be safely concluded that the various muscular respiratory and cardio vascular manifestations [such as arrhythmias, electrical abnormalities, hypertension, peripheral vascular collapse, congestive heart failure, pulmonary edema and morphologic changes in the myocard] of scorpion envenomation are a consequence of an activation-excitation of the nervous system.

However, studies from the seventies and early eighties indicated that certain scorpion venoms or their derived fractions are able to increase the contractility of skeletal muscle in a neuronally independent, non-medicated (direct) fashion attributed to the increase of sodium permeability of the muscle membrane [2]. Similarly an increase in the contractility of the cardiac muscle was indicated by various observations (summarized in [2]) and clearly demonstrated by Grupp et al. [3]. The latter have demonstrated that the positive inotropism induced by the venom of the yellow scorpion (which is toxic to mammals) to isolated hearts or their derived atrial and ventricular strips is only partially reduced by sympathetic blockers (such as propranolol). The remaining, slowly developing, sustained contractility was unaffected by any autonomous pharmacology, and therefore it represents a direct action on the cardiac muscle.

The non-toxic to mammals black scorpion (Buthotus judaicus, Buthinae, Scorpiones) is abundant in the vegetated populated regions of Israel [4]. Its venom: a) is practically non-toxic to mammals (180 μg/l gram mouse) unlike other Buthidae scorpions; b) possesses a strong toxicity to insects due to the presence of the excitatory (BjIT1) and the depressant (BjIT2)—Beta type insect selective neurotoxins; and: c) reveals a typical scorpion alpha toxin effect in an insect axon examined in a current and voltage clamp experiments [4].

### SUMMARY OF THE INVENTION

It is an object of the present invention to provide a polypeptide which has a positive inotropic effect and is non-toxic to mammals.

It is a further object of the invention to provide a pharmaceutical composition useful in the treatment of heart failure.

It is a still further object of the invention to provide a method for treatment of heart failure.

In a first aspect of the invention, there is provided a polypeptide which has a positive inotropic effect on cultured cardiomyocytes and is non-toxic to mammals.

The venom of the judean black scorpion (B. judaicus) has been found to contain a polypeptide non-toxic to mammals which strongly resembles the β depressant insect selective neurotoxins which are also non-toxic to mammals. This polypeptide has been found to have a positive inotropic effect, and will be at times referred to in the specification as “inotropin”. In the present specification, the term “non-toxic” refers to substances which do not have a pathological effect on mammals when coming into contact with them at concentration levels required for obtaining a therapeutic cardiotonic effect on the mammal. The polypeptide of the invention apparently acts directly on the muscle cells and not through the nervous system, since its positive inotropism was demonstrated on cultured and isolated cardiomyocytes which are not innervated (FIGS. 1, 2 and 4).

The N-terminal segment of Inotropin has been sequenced and the entire gene has been cloned. Inotropin has been found to have the following amino acid sequence:

(SEQ.ID.NO:1)

HDGYPKDSKGCKMTCITADDKFCNSICKGIGGKGECNWGVCWCTGVPNKN
DLWDSNNNKCGGK

and the following nucleic acid sequence:

```
(SEQ.ID.NO:2)
atgaagcgaattctggttttgatcgccttttcgttggtgttgataggagcagacgcgcat
  M K R I L V L I A F S L V L I G A D A H
gacggatatccaaaggacagcaagggatgcaagatgacttgtattacggcggatgataag
  D G Y P K D S K G C K M T C I T A D D K
ttctgcaatagtatatgtaaaggaatcggtggtaaaggcgaatgtaattggggggtttgt
  F C N S I C K G I G G K G E C N W G V C
tggtgtacaggagttccaaataaaaacgacctttgggattccaataataacaaatgtggt
  W C T G V P N K N D L W D S N N N K C G
gggaaatga
  G K -
```

The underlined segment is the leader peptide.

Synthetic or recombinant polypeptides may be produced by conventional means based on the above sequence.

As used herein, the twenty conventional amino acids and their abbreviations follow conventional usage (Immunology—A Synthesis, 2^{nd} Edition, E. S. Golub and D. R. Gren, Eds., Sinauer Associates, Sunderland Mass. (1991), which is incorporated herein by reference). Stereoisomers (e.g., D-amino acids) of the twenty conventional amino acids, unnatural amino acids such as α,α-disubstituted amino acids, N-alkyl amino acids, lactic acid, and other unconventional amino acids may also be suitable components for polypeptides of the present invention. Examples of unconventional amino acids include: 4-hydroxyproline, γ-carboxy-glutamate, ε-N,N,N-trimethyllysine, ε-N-acetyllysine, O-phosphoserine, N-acetylserine, N-formylmethionine, 3-methylhistidine, 5-hydroxylysine, ω-N-methyllarginine, and other similar amino acids and imino acids (e.g., 4-hydroxyproline). In the polypeptide notation used herein, the lefthand direction is the amino terminal direction and the righthand direction is the carboxy terminal direction, in accordance with standard usage and convention.

Similarly, unless specified otherwise, the lefthand end of single-stranded polynucleotide sequences is the 5′ end; the leffhand direction of double-stranded polynucleotide sequences is referred to as the 5′ direction. The direction of 5′ to 3′ addition of nascent RNA transcripts is referred to as the transcription direction; sequence regions on the DNA strand having the same sequence as the RNA and which are 5′ to the 5′ end of the RNA transcript are referred to as “upstream sequences”; sequence regions on the DNA strand having the same sequence as the RNA and which are 3′ to the 3′ end of the RNA transcript are referred to as “downstream sequences”.

In the present specification, the polypeptide of the invention includes homologous polypeptides in which one or more amino acids of SEQ.ID.NO:1 have been added to, deleted from or substituted by conservative substitution of homologous amino acids, on the condition that the resulting modified polypeptide substantially retains the insect selectivity (non-toxic to mammals) and positive ionotropic effect of inotropin. “Conservative substitution” refers to the substitution of an amino acid in one class by an amino acid of the same class, where a class is defined by common physicochemical amino acid side chain properties and high substitution frequencies in homologous proteins found in nature, as determined, for example, by a standard Dayhoff frequency exchange matrix or BLOSUM matrix. Six general classes of amino acid side chains have been categorized and include: Class I (Cys); Class II (Ser, Thr, Pro, Ala, Gly); Class III (Asn, Asp, Gln, Glu); Class IV (His, Arg, Lys); Class V (Ile, Leu, Val, Met); and Class VI (Phe, Tyr, Trp). For example, substitution of an Asp for another class m residue such as Asn, Gln, or Glu, is a conservative substitution.

Preferably, the resulting modified polypeptides have 90%, more preferably 95% or more sequence homology with inotropin.

The invention also relates to a DNA molecule encoding inotropin and comprising SEQ. ID. NO:2 with or without the leader sequence, a vector comprising the DNA molecule, and a host cell transformed with the vector. The invention further relates to nucleotide sequences which hybridize to SEQ. ID. NO:2 with or without the leader sequence under standard stringent hybridization conditions, for example: first cycle 94° C. for 4 min; thirty cycles 94° C. for 1 min, 55° C. for 30 sec, 72° C. for 45 sec, each; and last cycle 72° C. for 7 min.

A further aspect of the invention relates to a pharmaceutical composition comprising a polypeptide of the invention, and in particular a pharmaceutical composition for the treatment of heart failure by rendering a positive ionotropic effect on myocytes in general and cardiomyocytes in particular.

Another aspect of the invention relates to a pharmaceutical composition comprising crude Buthinae scorpion venom, and in particular a pharmaceutical composition for the treatment of heart failure by rendering a positive ionotropic effect on myocytes in general and cardiomyocytes in particular.

The polypeptides of the invention may be used to treat heart failure in general, and heart failure which involves reduced systolic function in particular. The delivery of the polypeptide may be performed, for example, by (a) Injection (parenteral)—intravenous or intramuscular; (b) Oral—via the digestive system. However, a polypetpide in order to be safely translocated through the gut should be “metabolically stabilized” to make it resistant to proteolytic enzymes; (c) Gene therapy.

In a preferred embodiment of the latter possibility, it would be advantageous if a patient could be provided with a long term, sustained delivery of a recombinant inotropic polypeptide according to the invention, for example, by the “Bio Pump” technology (edgenics Inc. Northridge, Calif. USA), which is based on transgenic-cultured cells (collected from the patient) engineered with DNA encoding inotropin. The above approach ensures a localized in vivo delivery of gene products in a controllable fashion through the employment of tissue specific promoters.

Recommended dosage of the pharmaceutical composition of the invention may be readily determined by the skilled man of the art. However a rough estimate may be based on a simple consideration related to human envenomation by a venomous scorpion such as the Yellow Israeli Scorpion. An average volume of a single sting of the Yellow Scorpion is about 0.55 μl which contains an average amount of about 57 μg of protein (˜10%), corresponding to about 2 μg of pure toxins (See A. Yahel-Niv and E. Zlotkin (1979) Comparative studies on venom obtained from individual scorpions by natural stings. Toxicon, 17:435-446). The above consideration leads to an approximate dose of 10 μg of the expressed cardiotonic polypeptide per day per adult patient.

The pharmaceutical compositions of the invention will optionally include pharmaceutically acceptable excipients as are well known in the art.

### BRIEF DESCRIPTION OF THE DRAWINGS

In order to understand the invention and to see how it may be carried out in practice, a preferred embodiment will now be described, by way of non-limiting example only, with reference to the accompanying drawings, in which:

FIGS. 1 and 2 are plots of fluorescent intensity caused by intracellular release of Ca⁺² ions in cardiomyocytes as a function of time (sec) and representing contractile force of resting cardiomyocytes treated with 100 μg/ml and 50μg/ml, respectively, of crude B. judaicus scorpion venom;

FIG. 3 shows an elution profile of a column chromatographical separation of he crude venom (0.7 mg) of the Black Scorpion by a reversed phase column on a analytical RP-VydacC18 column;

FIG. 4 is a plot of fluorescent intensity as a function of time (sec) of resting cardiomyocytes treated with 0.35 μg/ml of fraction IIIb from FIG. 3, which represents the effect of inotropin; and

FIG. 5 is a plot illustrating changes in the length of isolated cardiomyocytes derived from adult rats as a result of rhythmical contractions as a function of time.

### DETAILED DESCRIPTION OF SPECIFIC EMBODIMENTS

Materials and Methods

Crude venon: the venom of field collected Black Scorpions was electrically milked and lyophilized by well established methods [4].

Venom fractionation: The purification of the inotropic (SEQ ID NO: 1) factor was achieved by two successive steps of reversed phase column-chromatography on analytical columns (Vydac, USA 4.6×250 min) using a gradient of aceonitril—TFA 0.1%. The first step was applying 0.7 mg of the crude venom to an RPC 18 column. The fractions obtained on the C18 column were lyophilized and separated on an RPC8 column yielding pure components (data not shown).

Cardiac contractility—was monitored on cultured cardiomyocytes prepared from hearts of neonatal rats and their contractility was visualized by the aid of the intracellular calcium ion measurements using the indo 1 fluorescence ratio method as specified by Shneyways et al. (2001) J. Mol. Cell Cardiol., 33:1249.

The second expression of the increased mechanical contractility was demonstrated by an “optical method” which measures distances of rhythmical contractions of isolated cardiomyocytes derived from adult rats [Renaud et al (1986) Eur. J. Pharm. 120, 161].

Primary structure determination: the amino acid determination of the sequence of inotropin was performed in two steps: 1) the N-terminal sequence was determined by a micro-sequencing method on the reduced and alkylated protein; 2) the full sequence was determined by nucleotide sequencing of the clone of the toxin. The clone of the toxin was prepared by the RT-PCR protocol using degenerate primers based on the N-terminal amino acid sequence with the aid of SMART technology (Clontech, Palo Alto, Calif.). Briefly, total RNA was extracted from the scorpion venom gland segment (telson) by the method of Chomczynski and Sacci (Analytical Biochemistry (1987) 162:156-159) and reverse transcribed lo using SMART CDS primer and Powerscript Reverse3 Transcriptase (Clontech, Palo Alto, Calif.).

II. Experimental Results

A. Results with Crude Venom

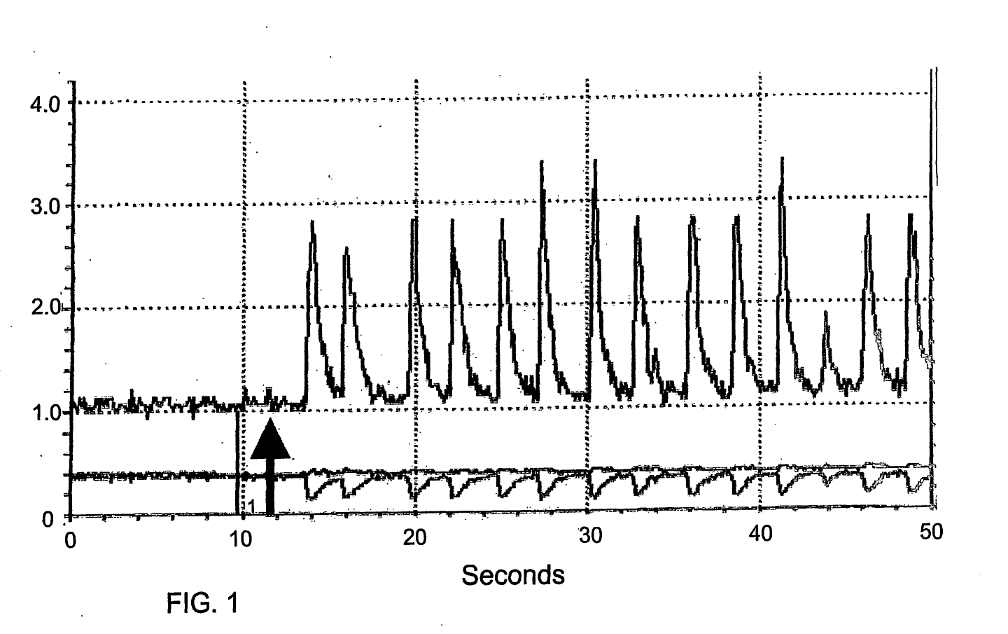

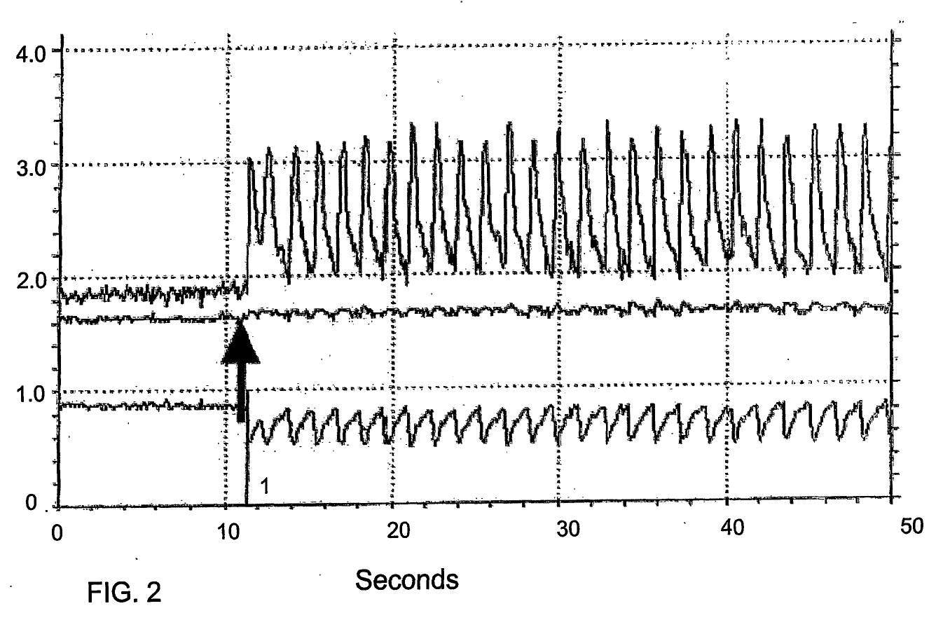

The crude, electrically collected and lyophilized venom of the Black Scorpion induces an obvious increase in the amplitude of contractions of cultured rat cardiomyocytes. The positive inotropic effect is exemplified in FIGS. 1 and 2. Application of the crude venom to “resting” (slightly contracting) cardiomyocytes induces a “train” of contractions of high amplitude.

B. Isolation of the Active Fractions from the Crude Venom

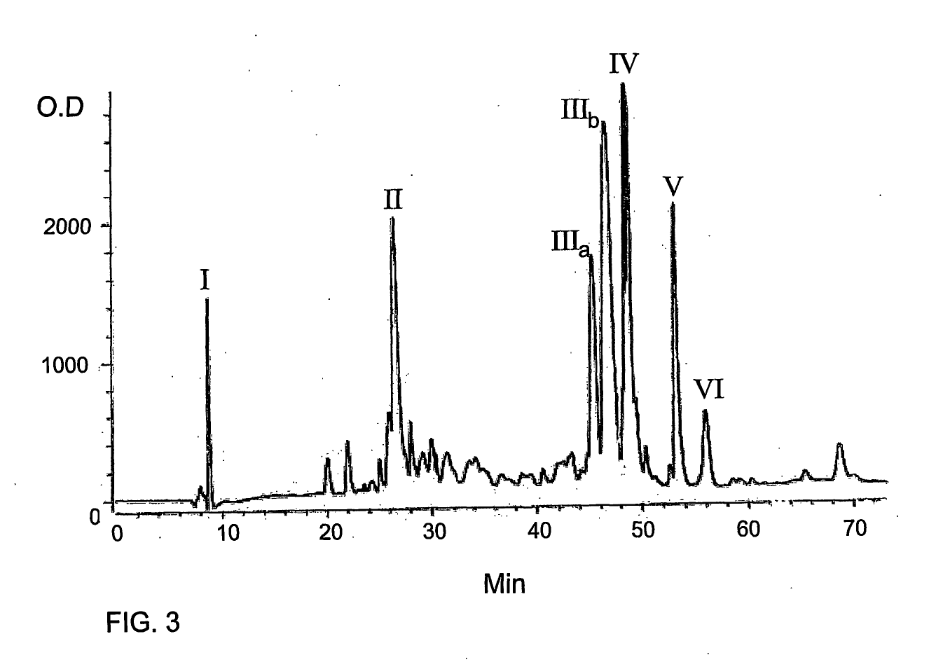

0.7 mg of lyophilized black scorpion venom were separated by HPLC on an analytical RP-VydacC18 column eluted by a gradient of acetonitril—TFA 0.1%. The resulting elution pattern is shown in FIG. 3. The elution yielded seven major fractions (I, II, IIIa, IIIb, IV, V and VI) and numerous smaller fractions.

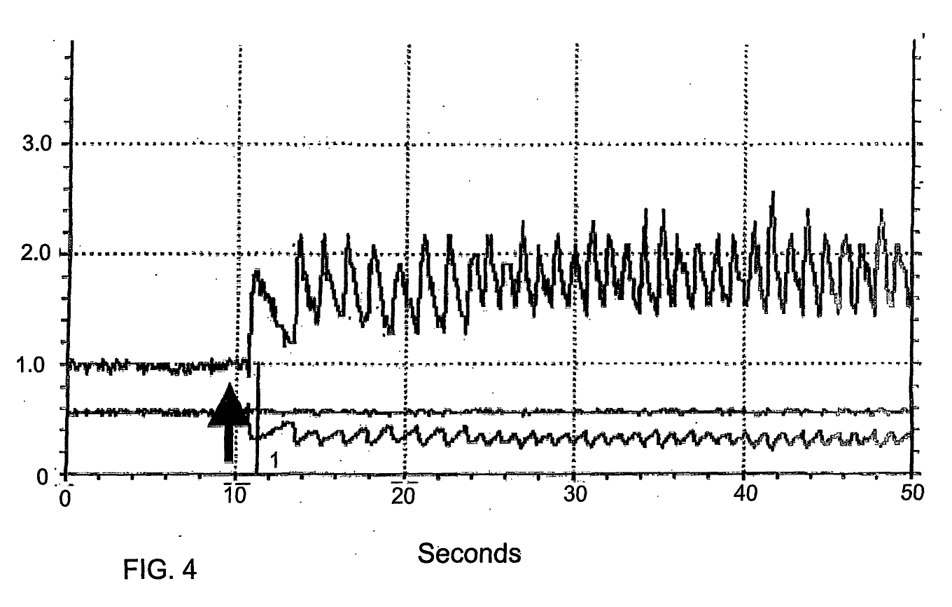

As shown in FIG. 4, fraction IIIb in doses about 200-300 times lower than the crude venom mimics its effect by inducing an augmented repetitive contractility of a “resting” (see above) cardiomyocyte. When higher doses of this fraction were applied they induced a transient rise in the level of the intracellular calcium followed by rhythmical contractions of increased amplitude and lower frequency (data not shown).

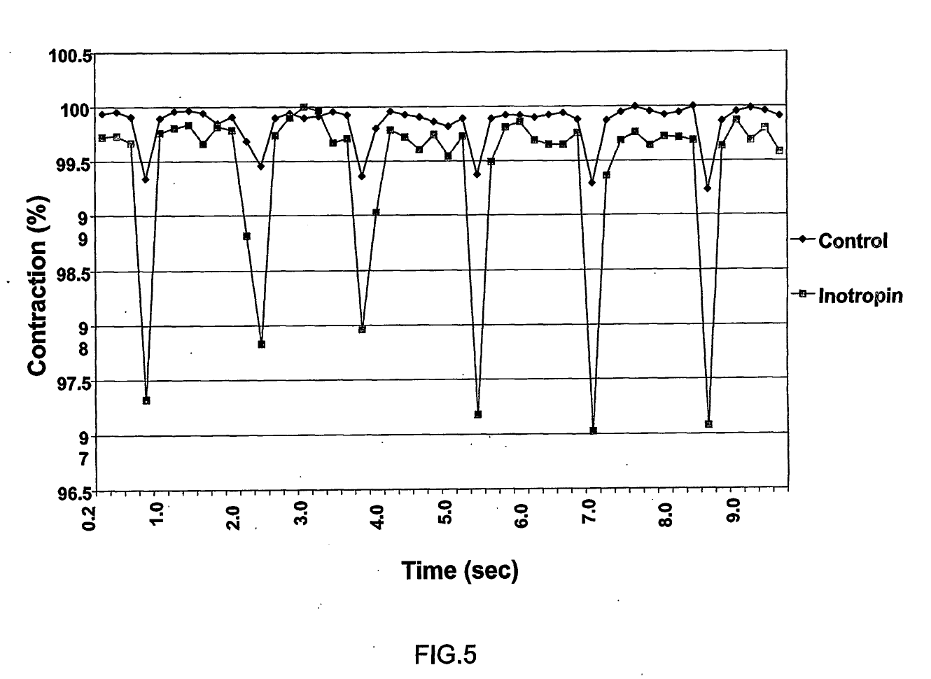

FIG. 5 shows the response of cardiomyocytes derived from a dissociated heart of an adult rat induced to contract by an electrical stimulation before adding inotropin and after addition of 0.35 μg/ml of inotropin. Note the obvious increase in contractility. This is a mechanical contractility which is optically monitored.

C. Amino Acid Sequence Determination of Inotropin

The full primary structure of inotropin isolated from fraction IIIb was determined by two approaches: a) microsequencing yielded 33 amino acids of the N-terminal section. The molecular weight as determined by mass spectral analysis was 6582 dalton; and b) by cloning and sequencing. Analysis of the amino acid sequences revealed an obvious resemblance (data not shown), to the scorpion venom Beta depressant insect selective neurotoxins which are non-toxic and non-lethal to mammals.

1-12. (canceled)

13. A pharmaceutical composition comprising a polypeptide selected from the following:
(a) A polypeptide which has a positive inotropic effect on cultured cardiomyocytes and is non-toxic to mammals.
(b) The polypeptide of (a) which has a direct inotropic effect on cardiac muscle and does not have an effect on the nervous system.
(c) The polypeptide of (a) whose amino acid structure resembles β-depressant insect selective neurotoxins.
(d) The polypeptide of (a) comprising SEQ. ID. NO:1.
(e) The polypeptide of (a) which is isolated from Buthinae scorpion venom.
(f) The polypeptide of (a) which is isolated from Buthinae judaicus scorpion venom.
(g) A synthetic positive inotropic polypeptide comprising an amino acid sequence having at least 90% homology to SEQ. ID. NO:1 and which is non-toxic to mammals.
(h) A synthetic positive inotropic polypeptide comprising an amino acid sequence having at least 95% homology to SEQ. ID. NO:1 and which is non-toxic to mammals.

14. A pharmaceutical composition according to claim 13 for the treatment of heart failure.

15. A method of treating heart failure in a patient comprising administrating a pharmaceutical composition according to claim 13 to said patient.

16. A pharmaceutical composition comprising crude Buthinae scorpion venom.

17. The pharmaceutical composition of claim 16 wherein said Buthinae scorpion is B. judaicus.

18. The pharmaceutical composition of claim 16 for inducing a positive inotropic effect in myocytes.

19. The pharmaceutical composition of claim 18 wherein said myocytes are cardiomyocytes.
